SEQUENCE LISTING (1 sequence)

SEQ ID NO: 1 - protein, 736 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFTAPKNVSDIIPRTEVEKAIRMSRSRINDAFRLNDNSLEFLGIQPTLGPPNQPPVS

(19) United States
(12) Patent Application Publication
(10) Pub. No.: US 2010/0316624 A1
(43) Pub. Date: Dec. 16, 2010

(54) TREATMENT OF FIBROSES AND LIVER DISORDERS

(76) Inventors: Hans Loibner, Wien (AT); Manfred Schuster, Schrick (AT)

Correspondence Address: GlaxoSmithKline;GLOBAL PATENTS -US, UW2220, P. O. BOX 1539, KING OF PRUSSIA, PA, 19406-0939, US

(21) Appl. No.: 12/808,859

(22) PCT Filed: Dec. 22, 2008
(86) PCT No.: PCT/AT2008/000472

§ 371 Date: Jun. 17, 2010

(30) Foreign Application Priority Data
Dec. 21, 2007 (EP) 07450239.4

(51) Int. Cl. A61K 38/48; A61K 31/7088; C12N 9/48; C07H 21/00; A61P 1/16; A61P 11/00; A61P 21/00; A61P 19/04; A61P 17/00; A61P 13/12

(52) U.S. Cl. 424/94.63; 514/44 R; 435/212; 536/23.2

(57) ABSTRACT

The present invention relates to ACE2 for the therapeutic treatment or prevention of a fibrosis or liver disorder.

The present invention relates to the field of treatment of fibroses and liver diseases, in particular inflammatory liver diseases.

Fibroses are diseases characterized by the formation of fibrotic tissue or tissue damage. This involves a pathological accumulation of connective tissue cells in the connective tissue itself or in an organ. The tissue of the organ in question becomes hardened, thereby resulting in scar tissue changes, which then in an advanced stage lead to restriction of the respective organ function.

Fibrosis is therefore understood to be excessive production of connective tissue in all human organs, the cause of which lies in overproduction of the proteins of the extracellular matrix, mainly collagen. The complex molecular regulation processes leading to this overproduction are understood only approximately. However, numerous causative factors have been identified so far, such as toxic substances, growth factors, peptide fragments and matrix proteins, hormones and the like, which stimulate fibroblasts such as myofibroblasts and stellate cells as target cells of the increased formation of matrix proteins.

The liver is an organ which has an extremely high level of metabolic activity while also being highly regenerative, i.e., capable of forming new liver cells and regenerating itself even at high levels of damage. There is marked tissue neogenesis in liver diseases, depending on the intensity, so there is also a high risk of formation of fibrotic tissue.

Huentelman et al. (Exp. Physiol. 90(5) (2005): 783-790)) describe the use of a lentiviral vector, encoding mouse ACE2 (lenti-mACE2), which has been used to investigate cardiac fibroses. The experimental model has been based on rats to which Ang II was administered with the help of implanted pumps. It has been demonstrated that fibrosis in the heart is caused by administration of Ang II and collagen production is also increased. Both effects are attenuated by transformation with the Ang II vector.

Herath et al. (Journal of Hepatology 47 (2007): 387-395)) describes a study on hepatic fibrosis models (BDL rats), in which fibroses were induced by a surgical procedure. This document does not relate to any treatment of this fibrosis and in particular no administration of Ang II but instead concerns only the observation of Ang II values and angiotensin (1-7) values.

In Warner et al. (Clinical Science 113 (2007): 109-118)) the effect of angiotensin II is summarized, in particular the effect on inflammation and control of wound healing. In chronic injuries, the ACE2 path of the RAS is naturally upregulated, in particular in the development of hepatic fibroses.

Diez-Freire et al. (Physiol Gen. 27 (2006): 12-19)) describe a preliminary study of the results of Huentelman et al. According to Diez-Freire et al., the ACE2 lentivirus transfection vector was also used to investigate the effect of ACE2 gene transfer on the blood pressure in particular.

Katovich et al. (Experiment. Physiol 90 (3) (2005): 299-305)) describe investigations of ACE2 on hypertension. It has been found that animals which express ACE2 (but transformed with the lentivirus vector) can be protected in particular from artificially induced hypertension caused by angiotensin II.

Huentelman M. (“HIV-1 Based Viral Vector Development for Gene Transfer to the Cardiovascular System” (2003) (dissertation)) describes vector systems for gene transfer to cardiovascular systems. ACE2 is discussed briefly on pages 11 and 12 therein, where ACE2-knockout mice are discussed specifically, with the finding that the ACE2 system is another system for regulating blood pressure which counteracts the ACE system. It is also proposed on page 71 that the lentivirus vector described by Huentelman et al. should be used for ACE2 transfection.

Kuba et al. (Curr. Opin. in Pharmac. 6 (2006): 271-276)) describe the protective function of ACE2 in ARDS animal models and SARS coronavirus infections because ACE2 is a critical SARS receptor.

Huentelman et al. (Regul. Peptides 122 (2004): 61-67)) describe the cloning of the water-soluble secreted form of ACE2. The truncated form of ACE2 was cloned in a lentivirus vector for transfection (“Lenti shACE2”). Cardiac cells or endothelial cells of the coronary arteries are mentioned as a target in particular. In comparison with membrane-bound ACE2, a higher secretion and thus increased ACE2 concentration in the circulation were thus found.

WO 2004/000367 describes ACE2 activation for treatment of diseases of the heart, lung and kidneys.

One goal of the present invention is to prevent developments which lead to fibroses and liver disease, to delay their advance and to treat fibroses and liver diseases.

The present invention therefore relates to a protein or a nucleic acid which encodes the protein, wherein the protein is ACE2, for therapeutic treatment or prevention of a liver disease or fibrosis. The present invention also relates to the use of an ACE2 protein or an ACE2-encoding nucleic acid for production of a pharmaceutical composition for treating or preventing liver disease or fibrosis.

The present invention has shown for the first time that the renin-angiotensin system exerts a significant influence on the pathological course of various organic diseases and that inactivation of same by therapeutic administration of ACE2 can relieve acute symptoms as well as help to cure chronic conditions. At this point it should be emphasized that in the case of an especially aggressive liver fibrosis model in particular, activation of stellate cells, which were originally considered to be responsible for the development of the fibrosis-induced organ dysfunctions of the liver, could be completely inhibited according to the present invention. Thus the pathological development could be stopped or even reversed. According to the present invention, this finding can be applied to a variety of fibrotic diseases due to the similarity in the pathological courses.

Angiotensin-converting enzyme 2 (ACE2) is an essential enzyme of the renin-angiotensin-aldosterone system that is expressed as a membrane-anchored glycoprotein on various organs such as the heart, kidneys, liver and lungs, but also blood vessels. ACE2 was discovered in 1997 as an ACE-homologous enzyme (GenBank Acc: BAB40370, encoded by nucleic acid having the gene sequence according to GenBank Acc.: AB046569). It was initially thought to have the same enzymatic activity as ACE (U.S. Pat. No. 6,989,363). Only later was it discovered that ACE2 has an entirely different mechanism from ACE and is in fact antagonistic (WO 2004/000367). ACE2 is a carboxypeptidase which cleaves numerous peptide substrates having markedly different selectivity and activity. ACE2 is also a cellular binding partner of SARS coronaviruses. Downregulation of ACE2 or administration of ACE2 to block viral receptors can therefore reduce the susceptibility of ACE2-presenting cells (WO 2006/122819). The functions described for ACE2 include in particular the conversion of Ang II to Ang 1-7, where the substrate and product of this reaction exhibit antagonistic properties. Ang II acts essentially with a vasoconstrictive and hypertensive effect. Ang 1-7 has a vasodilating effect and also has a protective effect in diseases of the kidneys, lungs and heart (WOO 2004/000367). The ACE2 product Ang 1-7 also inhibits ACE, the enzyme responsible for a production of Ang II, to a significant extent. The renin-angiotensin system plays an essential role in the pathology of liver diseases, specifically liver fibrosis. The presence of Ang II is responsible for profibrogenic effects in stellate cells of the liver (HSCs, hepatic stellate cells). It has been demonstrated that the expression and activity of ACE2 increase in patients suffering from chronic HCV infections. This is seemingly a protective mechanism, although it is not sufficient to initiate regeneration of the organ. An increase in ACE2 activity therefore leads to the goal.

According to the invention, the healing process can be accelerated by inhibiting the development of fibrosis and/or by preventing an exacerbation of the fibrosis. A prophylactic treatment is therefore possible with ACE2 or an ACE2-encoding nucleic acid. However, such a prophylactic treatment should not be understood in an absolute sense and instead should be understood only as a reduction in the risk of occurrence of a fibrosis or relieving the intensity of symptoms of a developing fibrosis. In particular the present invention relates to the treatment or prevention of progression of a fibrosis or liver disease. Prophylactic use is advisable in particular in risk patients who have a high probability of developing a fibrosis or liver disease (in comparison with healthy individuals), e.g., alcoholics or patients with a hepatitis C infection.

In preferred embodiments, the fibrosis is a local fibrosis of a tissue or organ. Such organ-specific fibroses include hepatic fibroses, pulmonary fibroses, connective tissue fibroses, in particular fibrosis of the muscle septa, renal fibrosis, and fibrosis of the skin, e.g., in combination with an inflammation-scleroderma. The fibrosis is preferably a fibrosis of an internal organ, e.g., the liver, kidneys, lungs, heart, stomach, intestines, pancreas, glands, muscles, cartilage, tendons, ligaments or joints. Cystic fibrosis or rheumatic fibrosis is a special form of fibrosis.

The fibrosis is preferably attributed to an excessive deposit of the components of the extracellular matrix, in particular proteins such as collagen. Collagen is a structural protein of the connective tissue, in particular the extracellular matrix. The formation of collagen, in particular in combination with the SMA (smooth muscle actin) marker correlates directly with the progression of fibrosis. According to the invention, especially effective inhibition of deposition of collagen by ACE2 has been observed.

In addition, based on the general mechanism, the treatment of chronic fibroses is also possible. In particular, the fibrosis may be caused by mechanical or chemical cell or tissue damage or wounds, cancer or tumors, infections, in particular pathogens such as viruses, bacteria or fungi, by implants, including organ implants as well as medications. Infections may be organ-specific, for example, such as hepatitis virus infection, in particular due to HCV. Other preferably fibrotic diseases, which may be treated with ACE2 or an ACE2-encoding nucleic acid according to the present invention, include, for example, primary or secondary fibroses, in particular fibroses caused by an autoimmune response, Ormond's disease, retroperitoneal fibrosis.

The liver is an organ which has an extremely high level of metabolic activity while also being highly regenerative, i.e., capable of forming new liver cells and regenerating itself even at high levels of damage. There is marked tissue neogenesis in liver diseases, depending on the intensity, so there is also a high risk of formation of fibrotic tissue The present application of ACE2 or an ACE2-encoding nucleic acid is thus suitable for treating liver diseases, in particular to prevent or treat fibrotic symptoms as a side effect or main indication. Furthermore, it has been demonstrated that ALT (alanine amino-transaminase), which is an indicator for liver function, may be significantly elevated by ACE2 treatment. The present invention is therefore suitable in particular for creating or preserving liver function in a liver disease. In specific embodiments, the liver disease is associated with liver damage or liver cell damage.

In special embodiments, the fibrosis or liver disease occurs concurrently with an inflammation, a hepatitis (inflammatory liver disease). Inflammations or infections of various organs or tissues often heal poorly at least in part and may lead to the formation of fibrotic tissue. An inflammatory reaction is a process in which defense cells are en route to an infection source, where they ensure the elimination of the cause. This inflammation may thus be caused by an infection, for example. Various mediator substances are released here, contributing toward the elimination of the inflammation while also creating the symptoms of inflammation. In a case of dysregulation of the response, these symptoms may cause the main damage and/or may be the source of the disease in general. Inflammations may also be induced artificially, e.g., in organ transplants which may ultimately result in rejection of the foreign organ. Likewise, inflammations may also be caused as a side effect of certain medications.

Expression of ACE2 is controlled by various stimuli. It has now been found that ACE2 is downregulated by the occurrence of inflammatory cytokines such as TNF-alpha, IFN-gamma or IL-4, which subsequently leads to various diseases and to an accumulation of Ang II in the respective compartments and to potentiation of the immune response that has been initiated. Cytokines serve essentially for communication among various types of cells of the immune system. One of the first steps of a nascent inflammation usually consists of the antigenic substances being taken up by antigen-presenting cells (APCs) and classified as foreign. In a further consequence, there is an initial output of inflammatory cytokines by the respective APCs, which then alarm additional cells of the immune system. This mechanism is highly regulated and controlled to initiate an immune response only when it is in fact justified and to turn it off again when the antigenic substance has been neutralized. It may nevertheless happen that once this immune response has been initiated, it gets out of control and turns against one's own body. The accumulation of Ang II, e.g., in various diseases of the kidneys, heart and lungs, causes a progressive inflammation and also an increased infiltration of the respective tissue by cells of the immune system and subsequently also a progression of the immune response. However, a key role here is always taken here by the cellular immune response as a response to a stimulus, which greatly overfulfills the primary purpose of neutralizing a foreign substance in a potentiating amplification cascade and subsequently damages the body.

The first step of the incipient immune response is to send out inflammatory signals in the form of cytokines. The main representatives of these include, for example, IL-4, IFN-gamma or TNF-alpha. Substances which have the property of suppressing or diminishing this cytokine expression after stimulation of the immune cell are usable therapeutic agents for attenuating an overshooting immune response. ACE2 expression drops sharply in the presence of these inflammatory cytokines on a cellular level, leading to a potentiation of the inflammation, especially due to an accumulation of Ang II, due to the decline in Ang 1-7 and due to the resulting lack of reduction in Ang II neogenesis. The sharp increase in Ang II concentration as a result promotes further potentiation of the inflammation due to the strong inflammatory properties of Ang II, subsequently leading to an even more marked attenuation of Ang II expression. To break out of this vicious cycle, according to the present invention, ACE2 is administered therapeutically and thus an accumulation of Ang II is prevented and the inflammation is suppressed: ACE2 directly reduces high Ang II titers, thereby diminishing the continuously exacerbating inflammation due to Ang II. Ang 1-7 is reformed and also diminishes the inflammation due to its anti-inflammatory action. In addition, Ang 1-7 limits the subsequent production of Ang II due to its property of inhibiting ACE. The subsiding inflammation causes the secreted cytokines to return to a normal level and causes a resumption of endogenous ACE2 expression, which then continuously ensures the degradation of Ang II and the formation of Ang 1-7 and again leads to a stable functional RAS. In the remaining course, a self-regulating stable equilibrium of the interacting components of the RAS is again established. A renewed administration of ACE2 may thus be omitted entirely if the original stimulus to the immune system has been neutralized. FIG. 5 shows a schematic diagram of the mechanisms mentioned above. Administration of ACE2 creates a way out of the exacerbating inflammation.

The data presented in the examples allow the following conclusions about the effect of ACE2 as an immune regulator. Inflammatory cytokines are released due to an antigenic stimulus. There is a loss of ACE2 expression in the presence of inflammatory cytokines. In the absence of ACE2, the proinflammatory peptide Ang II accumulates because it cannot be degraded by ACE2. In the absence of ACE2, the proinflammatory cytokine TNF-alpha also accumulates. ACE2 has anti-inflammatory properties and reduces the expression of inflammatory cytokines in lymphocytes. Therefore, therapeutic administration of ACE2 compensates for the lost endogenous ACE2 expression and can combat a nascent inflammation by lowering Ang II titers, by forming Ang 1-7 and by other effects. Therapeutic administration of ACE2 even makes it possible to reduce the Ang II titer back to the level of a healthy person and to restore regulation of the RAS accordingly, even in a case of severe sepsis with continuous LPS infusion. Therapeutic administration of ACE2 also makes it possible to lower the TNF-alpha titer back to the level of a healthy person in a case of severe sepsis with a continuous LPS infusion. The same effect has also been observed in a case of massive mechanical lung damage due to aspiration of meconium.

The protein is preferably recombinant ACE2. ACE2 sequences are sufficiently well-known and can be produced with no problem by introducing suitable ACE2-encoding vectors into expression systems, in particular eukaryotic systems. Such systems include, for example, mammalian cell lines such as CHO (Chinese Hamster Ovary) cells and NSO mouse cells or insect cells, e.g., Sf9. Such a vector may have certain cell-specific or general promoters for expression.

The protein (for which the ACE2 nucleic acids is also encoding) is preferably water-soluble ACE2, in particular without membrane domains. The human ACE2 sequence is given by SEQ ID No. 1:

MSSSSWLLLSLVAVTAAQSTIEEQAKTFLDKFNHEAEDLFYQSSLASWNY
NTNITEENVQNM-NNAGDKWSAFLKEQSTLAQMYPLQEIQNLTVKLQLQA
LQQNGSSVLSEDKSKRLNTILNTMS-TIYSTGKVCNPDNPQECLLLEPGL
NEIMANSLDYNERLWAWESWRSEVGKQLRPLYEEYVVLKNEMARANHYED
YGDYWRGDYEVNGVDGYDYSRGQLIEDVEHTFEEIKPLYEHLHAYVRAKL
M-NAYPSYISPIGCLPAHLLGDMWGRFWTNLYSLTVPFGQKPNIDVTDAM
VDQAWDAQRIF-KEAEKFFVSVGLPNMTQGFWENSMLTDPGNVQKAVCHP
TAWDLGKGDFRILMCTKVTMDDFLTAHHEMGHIQYDMAYAAQPFLLRNGA
NEGFHEAVGEIMSLSAATPKHLKSIGLLSPDFQEDNE-TEINFLLKQALT
IVGTLPFTYMLEKWRWMVFKGEIPKDQWMKKWWEMKREIVGVVEPVPHD
E-TYCDPASLFHVSNDYSFIRYYTRTLYQFQFQEALCQAAKHEGPLHKCD
ISNSTEAGQKLFNMLRLGKSEPWTLALENVVGAKNMNVRPLLNYFEPLFT
WLKDQNKNSFVGWSTDWSPYADQSIK-VRISLKSALGDKAYEWNDNEMYL
FRSSVAYAMRQYFLKVKNQMILFGEEDVRVANLK-PRISFNFFVTAPKNV
SDIIPRTEVEKAIRMSRSRINDAFRLNDNSLEFLGIQPTLGPPNQPPVS

The autologous signal sequence (underlined) is cleaved by the host cell for the removal. The inventive ACE2 protein therefore preferably comprises an ACE2 sequence corresponding to SEQ ID No. 1, starting in position 18. In additional embodiments, the ACE2 polypeptide does not have any transmembrane domains. These transmembrane domains are on the C terminus of SEQ ID No. 1. Therefore this is soluble ACE2. Especially preferred embodiments include soluble ACE2 polypeptides, whose polypeptide chain comprises of the amino acids SEQ ID No. 1 up to amino acid position 740 or enzymatically active fragments thereof. Another soluble ACE2 protein consists of amino acids 18-615 of SEQ ID No. 1.

The solubility of a protein is determined not only by its amino acid sequence but also by its folding and by post-translational modifications. Especially charged sugar structures which significantly increase the solubility of a protein and influence its pharmacological profile are included. The soluble section of ACE2 has seven N-glycosylation sites. Preferably at least 80% of the possible N-glycosylation positions are glycosylated and/or the ACE2 protein has a sugar content of greater than 10% (percent by weight of the total ACE2) or 11%, 12%, 13%, 14%, preferably great than 15% or 16%, 17%, 18%, 19%, in particular greater than 20% or 21%, 22%, 23%, 24%, or 25%.

Although human ACE2 is preferred for most embodiments, ACE2 from the mouse, rat, hamster, swine, primates or cattle is also possible. ACE2 is a universal enzyme in all mammals having the identical Ang II substrate. It may therefore also be used in foreign organisms. Thus, for example, humans, mice, rats, hamsters, swine, primates or cattle can be treated with the inventive protein (or its nucleic acid), regardless of the source of the ACE2.

According to the invention, a pharmaceutical composition comprising the ACE2 protein or an ACE2-encoding nucleic acid may be made available. Such compositions may comprise pharmaceutically acceptable salts thereof and additionally buffers, tonicity components or pharmaceutically acceptable vehicles. In particular, ACE2 nucleic acids may be provided in suitable therapeutic vector systems. Pharmaceutical vehicle substances are used to improve the tolerability of the composition and allow better solubility and better bioavailability of the active ingredients. Examples include emulsifiers, thickeners, redox components, starch, alcohol solutions, polyethylene glycol or lipids. The choice of a suitable pharmaceutical vehicle depends greatly on how it is administered. Liquid or solid vehicles may be used for oral administration; liquid final compositions are required for injections.

The medication to be used according to the invention preferably comprises buffer substances or tonic substances. The pH of the medication can be adjusted to physiological conditions by means of buffers, and fluctuations in pH can also be diminished, i.e., buffered. One example of this is a phosphate buffer. Tonic substances are used to adjust the osmolarity and may contain ionic substances such as inorganic salts, e.g., NaCl, or nonionic substances such as glycerol or carbohydrates.

The composition preferred for use according to the invention is prepared to be suitable for systemic, topical, oral or intranasal administration. These forms of administration of the medication according to the present invention allow a rapid and uncomplicated uptake. For example, for oral administration, solid and/or liquid medications may be taken directly or may be dissolved and/or diluted.

The medication to be used according to the invention is preferably prepared to be suitable for intravenous, intra-arterial, intramuscular, intravascular, intraperitoneal or subcutaneous administration. For example, injections or transfusions are suitable for this purpose. Administration directly into the bloodstream has the advantage that the active ingredients of the medication are distributed throughout the entire body and reach the target tissue rapidly.

The present invention is illustrated by the following, figures and examples without being limited to them.

### FIGURES

FIG. 1: Formation of collagen in the liver of wild-type and ACE2-knockout mice after 21 days of BDL, measured by Sirius Red staining (left) and mRNA assay (right) in comparison with a control group.

FIG. 2: Measurement of the SMA content in liver tissue (A) and its mRNA (B) in the liver of wild-type mice and ACE2-knockout mice after 21 days of BDL in comparison with a control group.

FIG. 3: Measurement of the SMA content in liver tissue (A) and its mRNA (B) in the liver of wild-type mice and ACE2-knockout mice after 21 days of BDL in comparison with a control group.

FIG. 4: BDL model in wild-type mice: measurement of the ALT content in serum specimens of untreated wild-type mice and those receiving ACE2 treatment.

FIG. 5: Schematic diagram of the restoration of functional RAS by ACE2 treatment. Red (+) arrows represent effects of the expanding immunoreactivity, whereas blue (−) arrows denote changes due to ACE2 therapy.

FIG. 6: ACE2-specific FACS analysis of Vero E6 cell preparations after incubation for 48 hours with 10 ng/mL IL-4 (A), IFN-gamma (B) or TRN-alpha (C) (curves with a peak in the middle) in comparison with an unstimulated control group (red curves with a peak on the right) and a control series (black curves with a peak on the left).

FIG. 7: Measurement of TNF-alpha in PBMC cultures supernatants 16 hours after stimulation with LPS, PHA and LPS+PHA, without ACE2 (black bars, left) or in the presence of ACE2 (grey bars, center) or ACE2 and Ang II (blue bars, right).

FIG. 8: Measured Ang II concentrations in an LPS-induced sepsis model in swine; blue curve: animals treated with APN 01 (rACE2); grey curve: animals treated with a placebo; grey curve (black dots): healthy animals after administration of APN 01.

FIG. 9: ACE2 activity measured in mice, swine and Rhesus macaques.

FIG. 10: Serum TNF-alpha concentration in an LPS-induced sepsis model in swine. Animals treated with ACE2 are shown in blue; animals treated with a placebo are shown in grey. TNF-alpha concentrations were standardized to the respective initial values at the start of treatment (100%).

FIG. 11: Serum TNF-alpha concentration in an ARDS model induced by aspiration of meconium in swine. Animals treated with ACE2 are shown in blue; animals treated with a placebo are shown in grey.

### EXAMPLES

### Example 1

### Liver Fibrosis Model, Importance of ACE2 in Liver Fibrosis

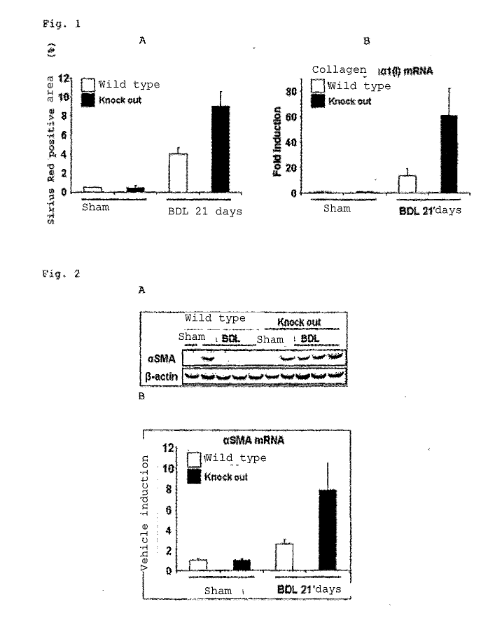

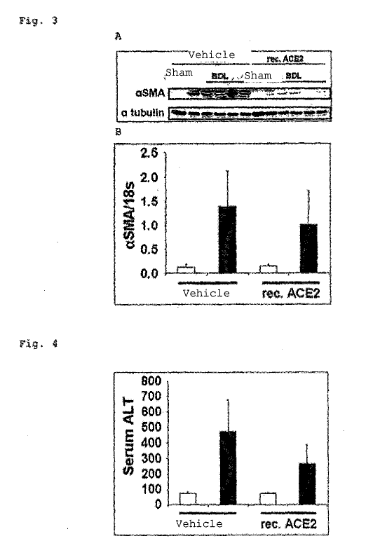

ACE2-knockout and ACE2 wild-type mice after ligation of the bile duct (bile duct ligation, BDL) were evaluated after 21 days and compared with sham control groups. Pathological examination of the liver revealed definitely elevated collagen deposits in the animals subjected to BDL (FIG. 1). The collagen deposit in the hepatic tissue was investigated by specific staining using Sirius red and was surprisingly found to be 2.5 times higher in ACE2-knockout animals than in the wild-type group (FIG. 1). The number of collagen-producing cells in the liver was determined by measuring SMA, which is a marker for activated stellate cells, by means of Western Blot and mRNA measurement. FIG. 2 shows the relationship between the lack of ACE2 activity and liver damage and shows clearly that the number of collagen-producing cells is definitely elevated.

This approach shows that there is a correlation between the absence of ACE2 and collagen deposits in a damaged liver. Collagen deposition is an important pathological symptom of progressive liver damage.

### Example 2

### Therapeutic Model

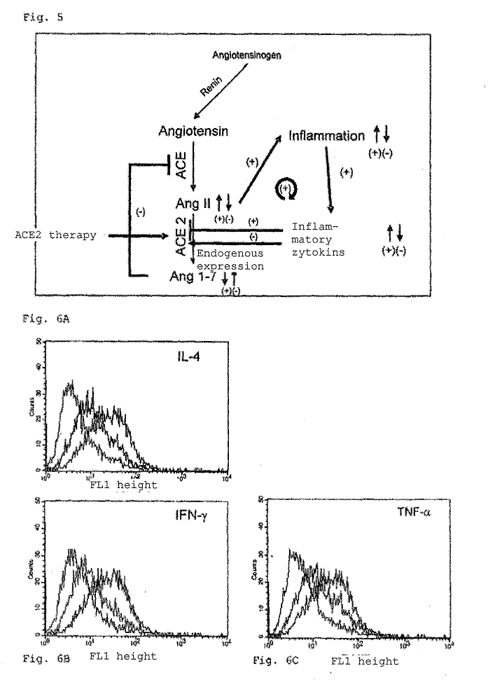

In a second approach, wild-type mice received a daily bolus injection of 2 mg/kg recombinant ACE2 intravenously after BDL for 14 days. After the end of the treatment, these animals were compared again with a control group that received only saline solution. FIG. 3 shows very clearly that the SMA concentration in tissue and thus the number of collagen-producing cells in the damaged liver tissue of the wild-type animals increase very significantly, but no SMA could be detected by Western Blot in the liver of Mice treated with ACE2. Analysis of the mRNA of SMA confirms this result. FIG. 4 shows the serum ALT concentration of the groups tested at the end of the experiment. It was also demonstrated here that ALT reached lower concentrations in the group treated with ACE2.

Both studies show very clearly that reduced ACE2 activity leads to an exacerbation of liver symptoms. A higher ACE2 activity reduces the number of collagen-producing cells and the accumulation of collagen in the tissue. Furthermore, a therapeutic effect of systemic administration of recombinant soluble ACE2 has been confirmed.

### Example 3

### Loss of ACE2 Expression in the Presence of Inflammatory Cytokines

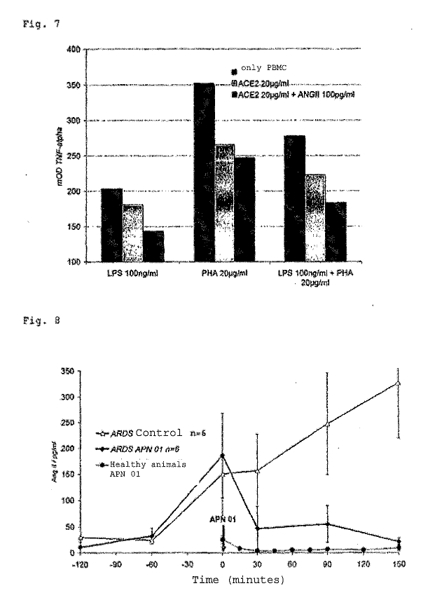

The renal cell line (Ceropithecus aethiops) Vero E6 expresses ACE2 as a membrane-anchored glycoprotein under the usual culture conditions. Vero E6 cells were incubated for 48 hours with 10 ng/mL IL-4, IFN-gamma or TNF-alpha, and changes in ACE2 surface expression were analyzed by FACS analysis using a polyclonal ACE2-specific goat antibody and a goat-specific FITC-labeled antibody. FIG. 6 shows the respective histograms. Table 1 summarizes the respective analysis. ACE2 expression is definitely reduced by incubation with IL-4, IFN-gamma or TNF-alpha. An ACE2 positivity of 51±3% was measured in unstimulated cells, but this was reduced to 28±2%, 22±1% and 39±2%, respectively, in comparison with an unstimulated control group after incubation of Vero E6 with 10 ng/mL IL-4, IFN-gamma or TNF-alpha for 48 hours.

TABLE 1
ACE2-specific FACS analysis, measured after incubation
of Vero E6 with 10 ng/mL IL-4, IFN-gamma or TNF-alpha for 48
hours in comparison with an unstimulated control group.

| Stimulation | IL-4 | IFN-gamma | TNF-alpha | Ø |
|---|---|---|---|---|
| Positivity | 28 ± 3% | 22 ± 1% | 39 ± 2% | 51 ± 3% |
| Negative controls | 5 | 2 | 4 | 6 |

### Example 4

### Attenuation of the Immunoreactivity of PBMCs

The effect of ACE2 on cytokine expression of stimulated PBMCs (peripheral mononuclear blood cells) is explained in this example. A PBMC preparation and thus the entire lymphocyte spectrum of the donor in the batch were used to allow the interaction of different lymphocytes. The PBMCs in whole blood from a healthy donor were separated by centrifugation. These cells were subsequently stimulated with strong immunogenic substances such as lipopolysaccharide (LPS, 100 ng/mL) and phytohemagglutinin (PHA, 20 μg/mL) and a combination of the two substances in the presence of Ang II, ACE2, and ACE2 with Ang II and then incubated for 16 hours at 37° C. The supernatants were tested for TNF-alpha and compared with a control batch, which was performed in the absence of ACE2 and peptides of RAS. The results of this experiment are plotted graphically in FIG. 3: Incubation with LPS with HPA in all cases induced secretion of TNF-alpha. The respective control batches, which were co-incubated without ACE2, showed the highest TNF-alpha concentrations (203, 352 and 278 mOD), each time after LPS, PHA and combination stimulation. In the presence of ACE2, the measured signal was definitely lower in all groups, reaching mOD values of only 181, 266, 233 in the respective groups. However, the measured TNF-alpha concentrations were the lowest in the presence of ACE2 and Ang II, reaching mOD levels of only 144, 247 and 183. These results show that the presence of ACE2 leads to a definitely reduced production of inflammatory cytokines, even if especially immunogenic substances such as LPS or PHA are used for stimulation. This confirms an anti-inflammatory effect of ACE2. Amazingly, the mechanism already functions in the absence of Ang II and is potentiated in its presence, which indicates a dual principal. A portion of the effect is achieved by Ang II and its degradation product Ang 1-7, but another portion evidently functions by way of degradation of one of the other ACE2 substrates and is not bound to Ang II that is present (FIG. 7).

### Example 5

### Restoring the Ang II Titer of the Healthy Body

This example demonstrates how administration of exogenous ACE2 brings a deregulated RAS back under control. APN 01 (recombinant soluble human ACE2) was therefore administered in a sepsis model in which sepsis is induced by administration of LPS. LPS was infused into the animals continuously, starting at the time −120 minutes, which led to a massive inflammation and subsequently to sepsis. Owing to the massive secretion of inflammatory cytokines, ACE2 expression ceased, which subsequently led to an accumulation of the inflammatory peptide Ang II (see FIG. 8).

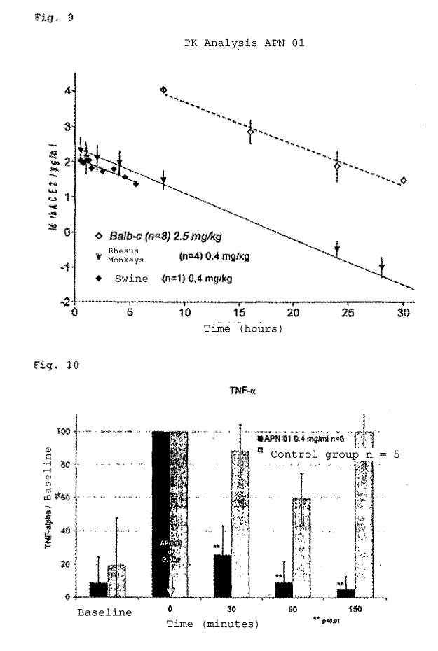

Starting at the time 0 minute, APN 01 was administered intravenously as a bolus in a dose of 400 μg/kg. There was an immediate drop in Ang II in the treated group, and the Ang II titer fluctuated within the following hour at the same level, which was also measured in the healthy animals. Furthermore, administration of APN 01 in the same dose to healthy animals also resulted in a brief decline in the Ang II titer, which also approximated the values of the healthy animals after another hour. However, animals treated with a placebo showed a further increase in Ang II level until the end of the experiment. This surprising phenomenon can be explained only by restoration of the upregulated RAS, because the active enzyme was available to the animals systemically until the end of the experiment (see FIG. 9). A half-life of approximately eight hours was measured.

### Example 6

### Attenuation of the Expression of Inflammatory Cytokines in Sepsis

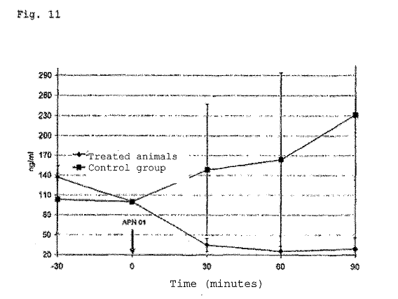

The following example demonstrates how the concentration of the inflammatory cytokine increases rapidly in a sepsis model in swine and drops back to the level of healthy animals after administration of ACE2. Starting at the time −120 minutes, LPS in a high dose was administered to the animals continuously, leading to a massive inflammation and subsequently leading to sepsis. Because of the massive secretion of inflammatory cytokines, this resulted in a reduction in ACE2 expression, which subsequently led not only to an accumulation of the inflammatory peptide Ang II but also the inflammatory cytokine TNF-alpha (FIG. 10). Starting at time 0 minute, either ACE2 in a dose of 0.4 mg/kg or buffer solution was administered as a bolus to the animals (six animals in the treated group, five animals in the control group). While LPS was still being administered continuously in the same high dose, the animals were observed for three more hours, while serum specimens were taken and analyzed for TNF-alpha. It was demonstrated that the TNF-alpha concentration in the control group remained elevated until the end of the experiment, whereas there was a definite reduction (p<0.001) in TNF-alpha concentration in the group treated with ACE2 already after a single dose of ACE2 and with continued administration of LPS. Despite massive sepsis, approximately the same values were again reached as those measured in healthy animals. TNF-alpha expression can therefore be reduced rapidly to the level of a healthy organism by administering ACE2 even in a very aggressive sepsis model, and a further potentiating inflammation can be stopped (FIG. 10).

### Example 7

Attenuation of Expression of all Inflammatory Cytokines after Local Mechanical Lung Damage.

In this example, the influence of systemically administered ACE2 on the expression of inflammatory cytokines was demonstrated in a lung damage model in swine. Fourteen animals were taken into account in this blinded, placebo-controlled study. All animals were subjected to aspiration of a 20% meconium solution three times in the first phase of the experiment, with comparable damage being induced in all animals on the basis of the hemodynamic parameters measured. In a second phase of the experiment, the therapeutic phase, recombinant soluble human ACE2 was administered intravenously as a bolus in a dose of 0.4 mg/kg to one-half of the animals. The other half received a physiological saline solution. Serum samples were taken at the times −30, 0, 30, 60, 90 and 150 minutes and used to measure the concentrations of the most important inflammatory cytokines. The time 0 was the starting point of the treatment, at which time all animals were already manifesting ARDS symptoms. As illustrated in FIG. 7, there is a very definite influence of administration of ACE2 on the serum concentration of TNF-alpha. Although this rises markedly to more than 230 ng/mL in the placebo group, it drops to less than 40 ng/mL within 30 minutes after administration in the treated group, approaching 25 ng/mL 90 minutes after administration.

1-14. (canceled)

15. The method of treating or preventing a fibrosis and/or a liver disease comprising administering ACE2 protein or an ACE2-encoding nucleic acid.

16. The method of claim 15 in which the fibrosis is a local fibrosis of a tissue or an organ.

17. The method of claim 15 in which the fibrosis is liver fibrosis, pulmonary fibroses, connective-tissue fibroses, muscle fibrosis, skin fibrosis or kidney fibroses, preferably liver fibrosis.

18. The method of claim 15 in which the liver disease leads to liver damage or liver-cell damage.

19. The method of treating of claim 15 in which the fibrosis or liver disease is associated with an inflammation, preferably hepatitis.

20. The method of claim 15 in which the fibrosis is liver disease is caused by an infection or a wound.

21. The method of claim 15 in which the ACE2 protein is a recombinant ACE2.

22. The method of claim 15 in which ACE2 protein is a water-soluble ACE2, in particular with no membrane domain.

23. The method claim 15 in which the ACE2 protein originates from a mammal, preferably a human being, a mouse, a rat, a hamster, a pig, a primate or cattle.

24. The pharmaceutical composition for the treatment of a fibrosis and/or liver disease comprising ACE2 or an ACE2-encoding nucleic acid.

25. The method of claim 15, wherein the method is a method of preventing development of a fibrosis and/or a liver disease before it occurs.
